(19) United States
(12) Patent Application Publication
(10) Pub. No.: US 2006/0058881 A1
(43) Pub. Date: Mar. 16, 2006

(54) Intervertebral disc nucleus implants and methods

(76) Inventor: Hai H. Trieu, Cordova, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/942,699

(22) Filed: Sep. 16, 2004

(51) Int. Cl. A61F 2/44

(52) U.S. Cl. 623/17.16

(57) ABSTRACT

Devices for anchoring spinal implants in an intervertebral disc space are provided. Spinal implants are also provided that are resistant to lateral deformation. The implants may include a flexible peripheral supporting band disposed circumferentially about an elastic body. Methods for anchoring spinal implants and methods for reducing deformation of spinal implants are also provided.

This application claims priority from U.S. patent application Ser. No. 10/842,103, filed May 10, 2004, which is a divisional application claiming priority from U.S. patent application Ser. No. 09/693,880, filed Oct. 20, 2000; and from U.S. patent application Ser. No. 10/253,453, filed Sep. 24, 2002, which is a divisional application claiming priority from U.S. patent application Ser. No. 09/650,525, filed Aug. 30, 2000 and issued Sep. 16, 2003 as U.S. Pat. No. 6,620,196; with all of said priority applications being incorporated herein by reference in their entirety.

### BACKGROUND OF THE INVENTION

The present invention relates generally to implants for replacing or augmenting an intervertebral disc, and more particularly to such implants that are resistant to lateral deformation.

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

Intervertebral discs may be displaced or damaged due to trauma or disease. Disruption of the annulus fibrosis allows the nucleus pulposus to protrude into the spinal canal, a condition commonly referred to as a herniated or ruptured disc. The extruded nucleus pulposus may press on the spinal nerve, which may result in nerve damage, pain, numbness, muscle weakness and paralysis. Intervertebral discs may also deteriorate due to the normal aging process. As a disc dehydrates and hardens, the disc space height will be reduced, leading to instability of the spine, decreased mobility and pain.

One way to relieve the symptoms of these conditions is by surgical removal of a portion or all of the intervertebral disc. The removal of the damaged or unhealthy disc may allow the disc space to collapse, which could lead to instability of the spine, abnormal joint mechanics, nerve damage, as well as severe pain. Therefore, after removal of the disc, adjacent vertebrae are typically fused to preserve the disc space.

Several devices exist to fill an intervertebral space following removal of all or part of the intervertebral disc in order to prevent disc space collapse and to promote fusion of adjacent vertebrae surrounding the disc space. Even though a certain degree of success with these devices has been achieved, full motion is typically never regained after such intervertebral fusions. Attempts to overcome these problems has led to the development of disc replacements. Many of these devices are complicated, bulky and made of a combination of metallic and elastomeric components and thus never fully return the full range of motion desired.

More recently, efforts have been directed to replacing the nucleus pulposus of the disc with a similar gelatinous material, such as a hydrogel. However, once positioned in the disc space, many hydrogel implants may migrate in the disc space and/or may be expelled from the disc space through an annular defect. Closure of the annular defect, or other opening, using surgical sutures or staples following implantion is typically difficult and, in some cases, ineffective. Moreover, such hydrogel implants may be subject to extensive deformation. Additionally, such hydrogel implants typically lack mechanical strength at high water content and are therefore more prone to excessive deformation, creep, cracking, tearing or other damage under fatigue loading conditions.

A need therefore exists for more durable nucleus pulposus or other spinal implants, including implants that are less resistant to deformation. The present invention addresses that need.

### SUMMARY OF THE INVENTION

Spinal implants are provided that are resistant to lateral deformation as they are restrained, or otherwise reinforced, by a flexible, peripheral supporting band. In one form of the invention, the implant includes an elastic body sized for introduction into the intervertebral disc space. The elastic body includes an upper surface and a lower surface for contacting adjacent vertebral endplates. A flexible peripheral supporting band is disposed circumferentially about the elastic body to reduce deformation of the body. At least a portion of the upper and lower surfaces of the elastic body are free of the supporting band. The implant, including the band, is sized to fit within an intervertebral disc space which is at least partially defined by an annulus fibrosis.

The implant that is resistant to lateral deformation may be used with or without a resorbable outer shell that aids in retaining the implant in a disc space. Additionally or alternatively, the implant may be used with or without an anchoring member that anchors the implant in a disc space.

One object of the present invention is to provide spinal implants that are more resistant to lateral deformation.

These and other objects and advantages of the present invention will be apparent from the descriptions herein.

### BRIEF DESCRIPTION OF THE FIGURES

FIG. 1 depicts a side view, in partial cross-section, of a nucleus pulposus implant, including an elastic body 15 and a supporting band 34, implanted in the intervertebral disc space of a disc.

FIG. 2 depicts a top, cross-sectional view of the nucleus pulposus implant of FIG. 1.

FIG. 3 depicts a side view, in partial cross-section, of a nucleus pulposus implant, including an elastic body 15 and a lateral support band 34, and further including a retaining strap for holding the lateral support band in position around the implant.

FIG. 4 shows a top, cross-sectional view of the nucleus pulposus implant of FIG. 3.

FIG. 5 shows a side view, in partial cross-section, of a nucleus pulposus implant, including an elastic body 15 surrounded by a supporting member 34, in the form of a band, wherein the supporting member is surrounded by an anchoring outer shell 30, implanted in the intervertebral disc space of a disc.

FIG. 6 depicts a side view of a cross-section of a nucleus pulposus implant, including an elastic body 15 surrounded by a supporting member 37, in the form of a jacket, wherein the supporting member is surrounded by an anchoring outer shell 30, implanted in the intervertebral disc space of a disc.

FIGS. 7A-7D depict various patterns of a supporting member of the present invention.

FIG. 8A depicts a side view of a cross-section of a nucleus pulposus implant including an elastic body 15 surrounded by a supporting member 34, taking the form of a band, that is further reinforced, or otherwise supported, by straps 420 and 430. The implant is surrounded by an anchoring outer shell 30 and is shown implanted in the intervertebral disc space of a disc.

FIG. 8B shows a top, cross-sectional view of the nucleus pulposus implant of FIG. 8A.

FIG. 8C depicts a side view of an alternative embodiment of a nucleus pulposus implant of the present invention that includes peripheral supporting band 34″ and securing straps 520, 530, 540 and 550 and is surrounded by an anchoring outer shell 30 and implanted in the intervertebral disc space of a disc.

FIG. 8D depicts a top, cross-sectional view of the nucleus pulposus implant of FIG. 8C.

FIG. 9 is a side view of a spinal implant system.

FIG. 10 depicts an end view of the system of FIG. 9, taken along line 10-10.

FIG. 11 depicts a side view of the spinal implant system of FIG. 9, implanted in an intervertebral disc space, that includes an anchoring component 10, an elastic body 100 and, optionally, a peripheral supporting band 101.

FIG. 12 depicts a side view of an alternative embodiment of a spinal implant system.

FIG. 13 depicts an end view of the system of FIG. 12, taken along line 13-13.

FIG. 14 depicts a side view of the system of FIG. 12 implanted in an intervertebral disc space.

FIG. 15A depicts a perspective view of a spinal implant that may be anchored with the anchoring devices described herein.

FIG. 15B depicts a side view of the implant of FIG. 15A.

FIG. 16 is a side view of a spinal implant reinforced with a flexible peripheral supporting band.

FIG. 17 depicts a top view of the implant of FIG. 16.

FIG. 18A shows the effect of imposing a load, represented by the darkened arrows, on the deformation of a spinal implant reinforced with a flexible supporting band. Top to bottom: no load; low load, moderate load; high load.

FIG. 18B is a graphical representation of the effect of imposing a load on the deformation of a spinal implant of FIG. 18A.

FIGS. 19A-19D depict alternative embodiments of a flexible peripheral supporting band of the present invention.

FIG. 20 depicts a side view of a spinal implant of the present invention that is reinforced, and otherwise supported, by peripheral supporting band 130′ and straps 134 and 135.

FIG. 21 shows a top view of the implant of FIG. 20.

FIG. 22 depicts a side view of an alternative embodiment of a spinal implant of the present invention, that includes a peripheral supporting band 130″ and securing straps 134′, 135′, 820, 830, 840 and 850.

FIG. 23 depicts a top view of the implant of FIG. 22.

FIG. 24 shows a cut-away view of an anchoring device implanted in an intervertebral disc space for anchoring implant 100 with a tension band 700 extending between vertebrae 107 and 109.

FIG. 25 depicts an anterior view of the device of FIG. 24.

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention relates to spinal implants that include an elastic body that is constrained and supported by a flexible supporting member, such as a peripheral supporting band. The spinal implants may be useful as nucleus pulposus replacements, partial or complete disc replacements, or may be useful in other disc reconstruction or augmentation procedures.

The band may advantageously have high resistance to hoop stress, and may thus function in a similar manner as the annulus fibrosis. More particularly, the hoop stress in the band preferably increases exponentially after some small, allowable initial deformation. Such implants may advantageously be used where the integrity of the annulus fibrosis has been negatively affected, or in other circumstances wherein increased support of an implant is needed.

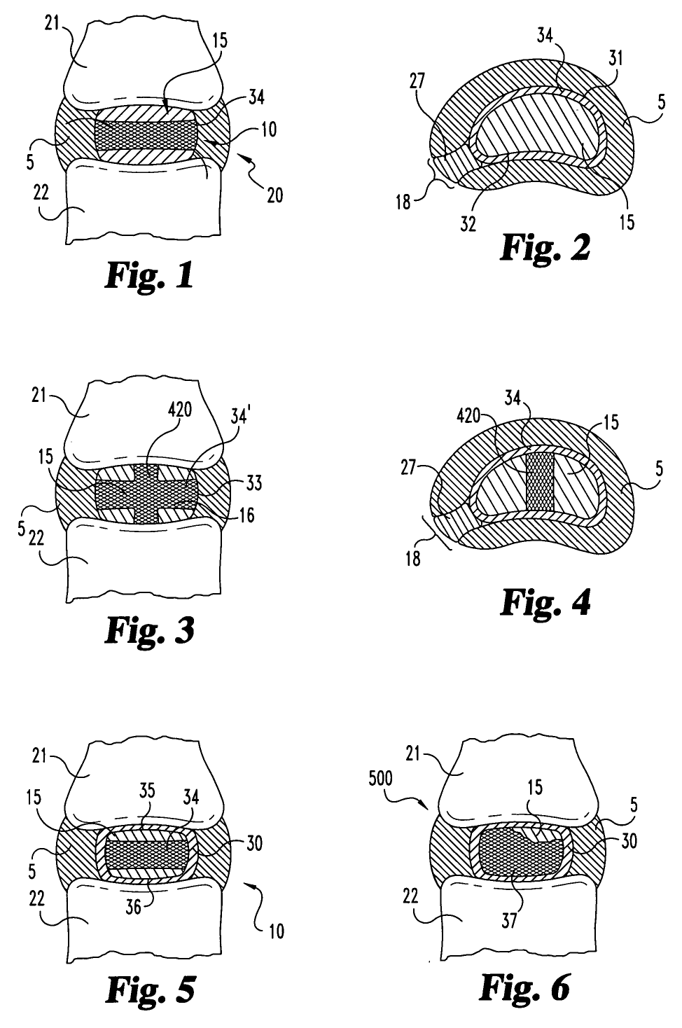

As disclosed above, in one aspect of the invention, a nucleus pulposus implant is provided that includes a load bearing elastic body sized for introduction into an intervertebral disc space, and a supporting member to control lateral expansion of the implant. As shown in FIGS. 1 and 2, prosthetic implant 10 includes a core load bearing elastic body 15 disposed in intervertebral disc space 20, between vertebral body 21 and 22. A peripheral supporting band 34 supports the body and protects against unwanted lateral deformation.

FIGS. 3 and 4 show an embodiment similar to the embodiment of FIGS. 1 and 2, but with an additional supporting strap that crosses above and below the implant. Such straps may be advantageous in preventing the peripheral supporting band described herein from slipping, or otherwise sliding off the implant. Said strap 420 extends along upper surface 35 of implant 15. As shown in the drawings, strap 420 is preferably connected, or otherwise attached, to peripheral supporting band 34′. The point of attachment may be any location that will secure the strap, including at the upper margins of the band, lower margins of the band, or any region between the upper and lower margins. Annular defect 18 may be filled with a plug 27 if desired to prevent migration of the nucleus from the annulus.

In some preferred embodiments the implant and supporting band are used in combination with an outer shell that facilitates secure implantation. In such embodiments elastic body 15 has an outer surface that may be in contact with, or even bonded to, an outer shell 30 that may advantageously be resorbable, or otherwise temporary. The outer surface of the outer shell preferably conforms to the shape of the intervertebral disc space 20, being in contact with annulus fibrosis 5, and may completely surround elastic body 15 as seen in FIGS. 5 and 6 and 8A-8D, although outer shell 30 may only partially surround elastic body 15. As an example, upper, lower and/or lateral voids surrounding elastic body 15 may be filled in by outer shell 30, as long as the elastic body is in some way anchored, or otherwise fixed in place, by the outer shell so as to prevent its expulsion from, or excessive migration in, the disc cavity. Thus, outer shell 30 may be configured to fill the aforementioned voids. Additionally, the inner surface of the outer shell preferably conforms to the shape of elastic body 15, and preferably bonds to the outer surface of elastic body 15 as discussed below. In preferred embodiments, the elastic core and the outer shell substantially fill the disc cavity as further discussed below.

Outer shell 30 not only provides for a properly fit implant 10 within intervertebral disc space 20 for maximum load-bearing, stress transfer, and bonding of the implant surface to the surrounding disc tissues for fixation against excessive migration, it also may seal an annular defect 18 for further resistance to migration and/or expulsion of the implant. Such sealing of the annular defect may also provide additional physical and mechanical support to the disc. Furthermore, the injectable outer shell material may provide intra-operative flexibility in fitting the core elastic body of implant 10 within the disc space as it may compensate for the differences in geometry and size between the disc space and the pre-formed core.

Outer shell 30 is preferably resorbable and, in such form, is preferably replaced with tissue, such as fibrous tissue and including fibrous scar tissue, that may aid in permanently confining the load bearing elastic body within the disc space. Accordingly, tissue may replace outer shell 30 after an appropriate passage of time, and thus surrounds elastic body 15. Although elastic body 15 may be confined within the disc space with the aid of tissue, body 15 is expected to have some mobility for normal biomechanics.

The dimensions of load bearing elastic body 15 may vary depending on the particular case, but elastic body 15 is typically sized for introduction into an intervertebral disc space. Moreover, elastic body 15 is preferably wide enough to support adjacent vertebrae and is of a height sufficient to separate the adjacent vertebrae. In order to provide long-term mechanical support to the intervertebral disc, the volume of elastic body 15 in the disc space should be at least about 50%, preferably at least about 70%, further preferably at least about 80% and more preferably at least about 90% of the volume of the entire disc space, the remaining volume occupied by outer shell 30. However, the volume of elastic body 15 may be as large as about 99% of the volume of the intervertebral disc space, and thus about 99% of the volume of implant 10. Accordingly, the volume of outer shell 30 may be at least about 1% of the volume of the implant, but may range from about 1% to about 50%. The appropriate size of implant 10 desired in a particular case may be determined by distracting the disc space to a desired level after the desired portion of the natural nucleus pulposus and any free disc fragments are removed, and measuring the volume of the distracted space with an injectable saline balloon. The disc volume can also be measured directly by first filling the disc space with a known amount of the outer shell precursor material.

Elastic body 15 may be fabricated in a wide variety of shapes as desired, as long as the body can withstand spinal loads and other spinal stresses. The non-degradable and preformed elastic body 15 may be shaped, for example, as a cylinder, or a rectangular block. The body may further be annular-shaped, and/or may have a spiral, or otherwise coiled, shape. Most preferably, elastic body 15 is shaped to generally conform to the shape of the natural nucleus pulposus, or may be shaped as further described below. Although elastic body 15 is shown as one piece in, for example, FIGS. 1-4, it may be made from one or several pieces.

Elastic body 15 may be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. The vulcanized rubber described herein may be produced, for example, by a vulcanization process utilizing a copolymer produced as described, for example, in U.S. Pat. No. 5,245,098 to Summers et al. from 1-hexene and 5-methyl-1,4-hexadiene. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable hydrophilic polymers include naturally-occurring materials such as glucomannan gel, hyaluronic acid, polysaccharides, such as cross-linked carboxyl-containing polysaccharides, and combinations thereof. The nature of the materials employed to form the elastic body should be selected so the formed implants have sufficient load bearing capacity. In preferred embodiments, a compressive strength of at least about 0.1 Mpa is desired, although compressive strengths in the range of about 1 Mpa to about 20 Mpa are more preferred.

Outer shell 30 may be formed from a wide variety of biocompatible, preferably elastic, elastomeric or deformable natural or synthetic materials, especially materials that are compatible with elastic body 15. The outer shell materials preferably remain in an uncured, deformable, or otherwise configurable state during positioning of the elastic body in the interverterbral disc space, and should preferably rapidly cure, become harder or preferably solidify after being introduced into the intervertebral disc space, or, in other embodiments, prior to positioning of the elastic body in the intervertebral disc space. In preferred embodiments, the outer shell materials may remain deformable after they harden or otherwise solidify. Suitable materials that may be used to form the outer shell include tissue sealants or adhesives made from natural or synthetic materials, including, for example, fibrin, albumin, collagen, elastin, silk and other proteins, polyethylene oxide, cyanoacrylate, polylactic acid, polyglycolic acid, polypropylene fumarate, tyrosine-based polycarbonate and combinations thereof. Other suitable materials include demineralized bone matrix. These precursor materials may be supplied in liquid, solution or solid form, including gel form.

Elastic body 15 may include a variety of surface features on outer surface 16, including chemical modifications and surface configurations, to provide surface features that advantageously improve the bonding between outer surface 16 of the elastic body and inner surface 32 of outer shell 30. In one form of the invention, outer surface 16 is chemically modified utilizing, for example, chemical groups that are compatible with the materials used to form outer shell 30. Suitable chemical modifications include, for example, surface grafting of reactive functional groups, including hydroxyl, amino, carboxyl and organofunctional silane groups. The groups may be grafted by methods known to the skilled artisan. Other modifications include pre-coating with a primer, preferably one that is compatible with the outer shell material, such as a layer of adhesive, sealing or other materials used for forming the outer shell described above.

In certain forms of the invention, the implant may include only elastic body 15 having one or more of the outer surface features as described above, without the outer resorbable shell. The surface features are expected to provide a certain level of fixation to the surrounding tissues for improved resistance to migration and/or expulsion.

In yet other forms of the invention, the implant may include an elastic body that is surrounded by a supporting, or otherwise constraining, member wherein the supporting member is surrounded by a resorbable shell as described herein. Referring now to FIG. 5, implant 400 includes a load bearing elastic body 15 that is surrounded by a supporting member 34. In one form, supporting member 34 may be a preferably flexible, peripheral supporting band that is disposed circumferentially about elastic body 15 as seen in FIG. 5, leaving upper and lower surfaces 35 and 36, respectively, of elastic body 15 free from the supporting band.

As seen in FIG. 5, portions of upper and lower surfaces 35 and 36, respectively, of elastic body 15 are exposed to directly contact outer shell 30. This exposure minimizes the amount of material needed to construct the supporting member, yet still effectively provides, for example, lateral support. Although the amount of the upper and lower surfaces of elastic body 15 that are exposed may vary, typically at least about 50%, preferably at least about 70%, more preferably at least about 80% and most preferably at least about 90% of the surfaces are exposed.

In yet another embodiment shown in FIG. 6, nucleus pulposus implant 500, that includes elastic body 15 as described above, is reinforced with supporting member 37, which takes the form of a jacket. The jacket preferably completely surrounds elastic body 15.

Suitable supporting members, including reinforcing outer bands, covers, or other jackets, may be formed from a wide variety of biocompatible polymers, metallic materials, or combination of materials that form a strong but flexible support to prevent excessive deformation, including lateral (horizontal) deformation, of the core under increasing compressive loading. Suitable materials include non-woven, woven, braided, or fabric materials made from polymeric fibers including cellulose, polyethylene, polyester, polyvinyl alcohol, polyacrylonitrile, polyamide, polytetrafluorethylene, polyparaphenylene terephthalamide, and combinations thereof. Other suitable materials include non-reinforced or fiber-reinforced elastomers such as silicone, polyurethane, copolymers of silicone and polyurethane, polyolefins, including polyisobutylene and polyisoprene, neoprene, nitrile, vulcanized rubber, and combinations thereof. In a preferred form of the invention, a combination, or blend, of silicone and polyurethane is used. Furthermore, the vulcanized rubber is preferably produced as described above for the nucleus pulposus implants. Supporting members 34 and 37 are advantageously made from a porous material, which, in the case of an elastic body made from a hydrogel, or other hydrophilic material, allows fluid circulation through the elastic core body to enhance pumping actions of the intervertebral disc. Supporting members may further be formed from carbon fiber yarns, ceramic fibers, metallic fibers or other similar fibers as described, for example, in U.S. Pat. No. 5,674,295.

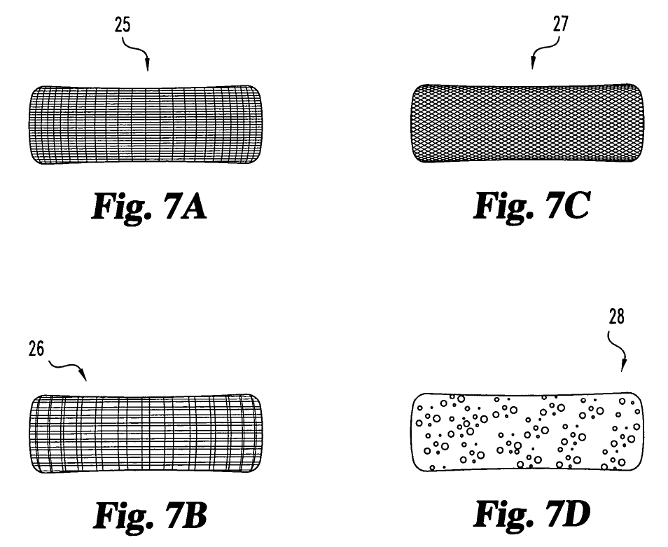

FIGS. 7A-7D show supporting bands of various patterns, typically made from various braided materials (bands 25, 26 and 27), or porous materials (band 28), as described above. It is also understood the jackets may also be formed of such patterns. It is realized that the braided materials may also be porous.

Supporting members 34 and 37 preferably decrease lateral deformation, compared to deformation of an implant without the supporting member, as desired. Supporting members 34 and/or 37 may, for example, decrease lateral deformation by at least about 20%, preferably at least about 40%, more preferably by at least about 60% and most preferably by at least about 80%. An implant, such as one that includes an elastic body, having such a supporting member will be flexible and otherwise resilient to allow the natural movements of the disc and provides shock absorption capability at low to moderate applied stress, but will resist excessive deformation for disc height maintenance under high loading conditions. As described herein in the case of a lumbar disc, for example, low applied stress includes a force of about 100 Newtons to about 500 Newtons moderate stress includes a force of about 500 Newtons to about 1000 Newtons, and high loading conditions, or high stress, includes a force of above about 1000 Newtons. In preferred forms of the invention, the supporting member is flexible, in that it may be folded, or otherwise deformed, but is substantially inelastic, so that the implant is more fully reinforced or otherwise supported.

The elastic body may be covered by the jacket supporting member, or the band supporting member may be wrapped around the circumference of the elastic body. In a form of the invention wherein the elastic body is formed from a hydrogel, or similar hydrophilic material, the hydrogel may be dehydrated a desired amount prior to being covered by the jacket, or prior to wrapping the band around the circumference of the hydrogel body. The hydrogel elastic body may be exposed to saline outside of the body, or may be inserted into the disc space wherein it will be exposed to body fluids in situ, and the body will absorb water and swell. In reference to the peripheral band supporting member, the swelling or expansion of the hydrogel elastic body in the horizontal direction is controlled by the amount of slack designed in the band. After the limited allowable horizontal expansion is reached, the elastic body is forced to expand mostly in the vertical direction until reaching equilibrium swelling under the in vivo load. As the upper and lower surfaces of the elastic body are not substantially constrained, the vertical expansion is mainly controlled by the applied stress and the behavior of the hydrogel material.

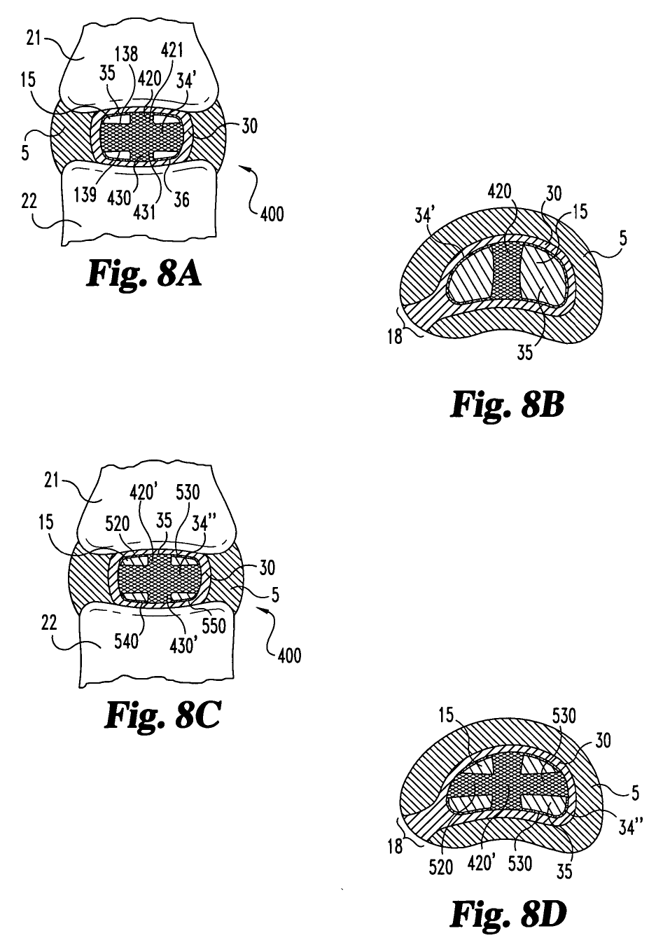

In yet other forms of the invention, an implant reinforced with a peripheral supporting band as described above that is surrounded by a resorbable outer shell may be further reinforced with one or more straps. The straps may be advantageous in preventing the peripheral supporting band described herein from slipping, or otherwise sliding off the implant. Referring now to FIGS. 8A and 8B, at least one strap 420 extends along upper surface 35 and at least one strap 430 extends along lower surface 36 of elastic body 15 of implant 400. Ends 421 of strap 420 and ends 431 of strap 430 are each preferably connected, or otherwise attached, to peripheral supporting band 34′. The point of attachment may be any location that will secure the strap, including at the upper margins 138 of the band, lower margins 139 of the band or any region between the upper and lower margins. Although two straps 420 and 430 are shown extending along upper surface 35 and lower surface 36, respectively, in FIGS. 8A and 8B, one continuous strap may be utilized that extends completely around the implant, or the strap utilized may be in one, two or multiple pieces, as long as the combination of straps are sufficient to prevent excessive slipping and or sliding of the supporting band. Furthermore, more than one strap may extend along upper surface 35 and more than one strap may extend along lower surface 36 of elastic body 15, as seen, for example, in FIGS. 8C and 8D of implant 500, wherein straps 520, 530, 540 and 550 are shown attached, or otherwise connect to supporting member 34″. It is realized that the straps may be present in one or more pieces. For example, straps 520 and 530 may form a single strap, as may straps 540 and 550, or may all combine to form a single strap.

In other aspects of the invention, kits designed for forming the intervertebral disc nucleus pulposus implants that include the outer shell described above are provided. In one form, a kit may include a load bearing elastic body as described above, along with a container of material to form the outer, preferably resorbable, shell. The material may be selected from the materials as described above. Moreover, the container that houses the material that forms the shell may be made from a wide variety of materials that are compatible with the outer shell material, including glass and plastic. The kit may further include a supporting member, such as a supporting band, jacket or other outer cover as described above. Generally, the kits include sterile packaging which secures the kit components in spaced relation from one another sufficient to prevent damage of the components during handling of the kit. For example, one may utilize molded plastic articles known in the art having multiple compartments, or other areas for holding the kit components in spaced relation.

The implants formed from an elastic material, including the outer shell and/or the supporting band, may advantageously deliver desired pharmacological agents. The pharmacological agent may include a growth factor that may advantageously repair the endplates and/or the annulus fibrosis. For example, the growth factor may include a bone morphogenetic protein, transforming growth factor-β (TGF-β), insulin-like growth factor, platelet-derived growth factor, fibroblast growth factor or other similar growth factor or combination thereof having the ability to repair the endplates and/or the annulus fibrosis of an intervertebral disc.

The growth factors are typically included in the implants in therapeutically effective amounts. For example, the growth factors may be included in the implants in amounts effective in repairing an intervertebral disc, including repairing the endplates and the annulus fibrosis. Such amounts will depend on the specific case, and may thus be determined by the skilled artisan, but such amounts may typically include less than about 1% by weight of the growth factor. The growth factors may be purchased commercially or may be produced by methods known to the art. For example, the growth factors may be produced by recombinant DNA technology, and may preferably be derived from humans. As an example, recombinant human bone morphogenetic proteins (rhBMPs), including rhBMP 2-14, and especially rhBMP-2, rhBMP-7, rhBMP-12, rhBMP-13, and heterodimers thereof may be used. However, any bone morphogenetic protein is contemplated including bone morphogenetic proteins designated as BMP-1 through BMP-18.

BMPs are available from Genetics Institute, Inc., Cambridge, Mass. and may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al. All bone morphogenic proteins are contemplated whether obtained as above or isolated from bone. Methods for isolating bone morphogenetic protein from bone are described, for example, in U.S. Pat. No. 4,294,753 to Urist and Urist et al., 81 PNAS 371, 1984.

In other forms of the invention, the pharmacological agent may be one used for treating various spinal conditions, including degenerative disc disease, spinal arthritis, spinal infection, spinal tumor and osteoporosis. Such agents include antibiotics, analgesics, anti-inflammatory drugs, including steroids, and combinations thereof. Other such agents are well known to the skilled artisan. These agents are also used in therapeutically effective amounts. Such amounts may be determined by the skilled artisan depending on the specific case.

The pharmacological agents are preferably dispersed within the hydrogel, or other hydrophilic, implant for in vivo release, and/or, with respect to the implants with the resorbable outer shell and/or with a supporting band, may be dispersed in the outer shell or band. The hydrogel may be cross-linked chemically, physically, or by a combination thereof, in order to achieve the appropriate level of porosity to release the pharmacological agents at a desired rate. The agents may be released upon cyclic loading, and, in the case of implants including a resorbable outer shell, upon resorption of the shell. The pharmacological agents may be dispersed in the implants by adding the agents to the solution used to form the implant, by soaking the formed implant in an appropriate solution containing the agent, or by other appropriate methods known to the skilled artisan. In other forms of the invention, the pharmacological agents may be chemically or otherwise associated with the implant. For example, the agents may be chemically attached to the outer surface of the implant.

The implants described herein may have embedded therein small metal beads or wire for x-ray identification.

Methods of forming and implanting the nucleus pulposus implants described herein are also provided. In one form of the invention, with respect to implant 10 described above having the anchorable outer shell 30, implant 10 may be formed by first forming elastic body 15 and then forming the outer shell. Methods of forming elastic body 15 are well known in the art.

For example, if the elastic body is made of elastomeric materials, such as powdered elastomers including, for example, styrene-ethylene/butylene block copolymers, the powdered elastomer may be placed into an appropriate mold and may be compressed and heated to melt the powder. The mold is then cooled to room temperature. If the elastic body is made from a hydrogel, such as a polyvinyl alcohol, the polyvinyl alcohol powder may be mixed with a solvent, such as, for example, water or dimethylsulfoxide, or combinations thereof, and heated and shaken until a uniform solution is formed. The solution may then be poured into a mold, such as a rubber mold, and may be cooled at an appropriate temperature, such as about 0° C. to about −80° C., for several hours to allow for crystallization. After cooling, the hydrogel can be partially or completely hydrated by soaking and rinsing with water but, in certain preferred embodiments, may remain dehydrated so that it may be inserted through a smaller aperture in the annulus fibrosis.

Prior to positioning the implant in the interverterbral disc space, an incision may be made in the annulus fibrosis, or one may take advantage of a defect in the annulus, in order to remove the natural nucleus pulposus and any free disc fragments within the intervertebral disc space. The disc space is then distracted to a desired level. The size of the disc space may then be determined using x-ray and/or other measurement techniques to determine disc dimensions and/or volume. For example, disc height and implant length and width may be derived from coronal and sagital plane radiographs. Additionally or alternatively, disc volume may be determined by filling the vacated disc space with saline, or by inflating a balloon within the disc space. The volume of the saline or balloon gas corresponds to the volume of the disc space.

Once formed, and after preparing the disc space for receiving the implant, elastic body 15 may be implanted into the intervertebral disc space utilizing devices well known in the art and as described in U.S. Pat. Nos. 5,800,549 and 5,716,416. If the outer shell precursor material was already placed in the intervertebral disc space, excess precursor material may flow out of the disc space. This excess material should be promptly removed before it sets or otherwise cures. The outer shell material may be injected, or otherwise introduced, into the disc space utilizing devices that are well known in the art, such as syringes, sealant/caulk guns, automatic liquid injectors, and applicators that include, for example, two separate syringes which allow for simultaneous mixing of the components in a static mixer and delivery to the site, and may be injected either prior to or after introduction of the implant into the disc space. Whether the outer shell material is introduced prior to or after introduction of the implant into the disc space, the distractor is then removed, any excess precursor material seeping out of the disc space is removed and the precursor material within the disc space is cured to form the outer shell. It is noted that the elastic body may already be surrounded by the outer shell, which may be in a partially or fully hardened state but preferably remains deformable, prior to introducing the elastic body into the intervertebral disc space.

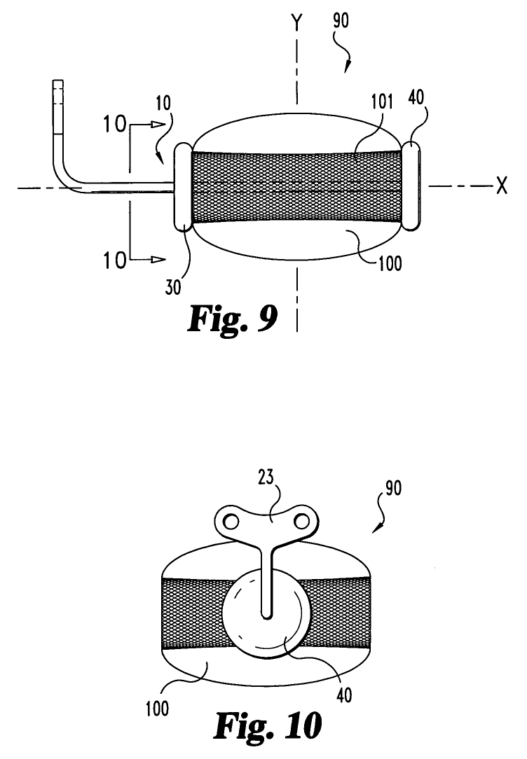

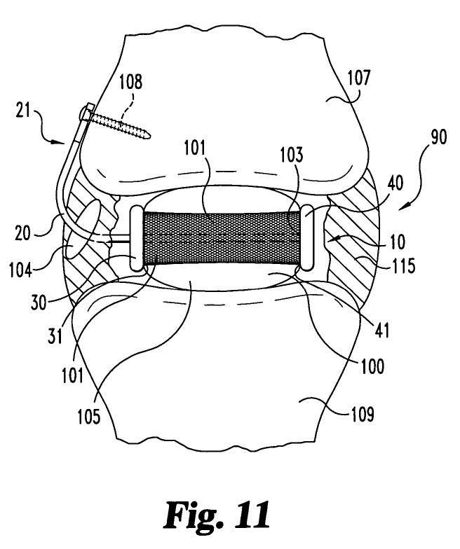

In the embodiments shown in FIGS. 9-14, spinal implant system 90 includes a spinal implant 100, and may include a spinal implant anchoring device 10. Inner surface 31 and 41 of securing members 30 and 40, respectively, abut outer surface 105 of implant 100. As seen in FIG. 11, anchoring rod 20 may extend through aperture, or other defect, 104 in annulus fibrosis 115 so that the first end 21 of anchoring device 10 may be anchored to upper vertebra 107 with a bone screw 108. First end 21 may, of course, be anchored to lower vertebra 109, or may be secured to both vertebrae 107 and 109 if first end 21 is appropriately configured as discussed above. The longitudinal axis X of the rod may extend parallel to the longitudinal axis Y of the implant, but may extend through the implant in a wide variety of directions, as long as the rod functions to anchor the implant in the disc space. Furthermore, the anchoring rod preferably extends at least partially through the implant, but may extend completely through the implant, entering one location, such as an end, and exiting another location, such as another end, including an opposing end. In preferred forms of the invention, implant 100 may include a peripheral supporting band 101 as further described below to provide further lateral support for the implant, as well as to improve the strength of the implant. In one form of the invention, band 101 may have apertures, or other openings therethrough, on opposing sides of the band which are in contact with the securing member to allow the anchoring rod of the anchoring component, or device, to be placed therethrough. Moreover, implant 100 further includes a channel 103 extending therethrough through which the anchoring rod may be disposed. The implant is preferably molded such that the channel is formed during the molding process. However, the channel may be formed after formation of the implant in a variety of ways, including drilling to form a channel having a desired shape with an appropriate drill bit.

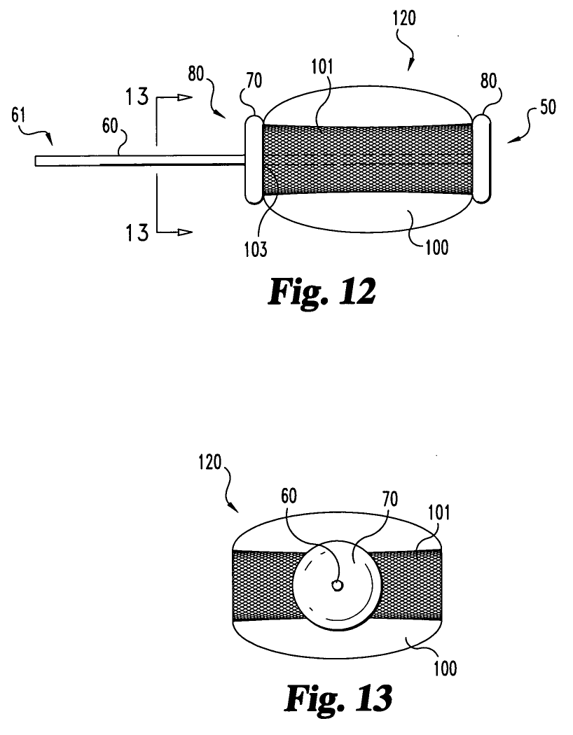

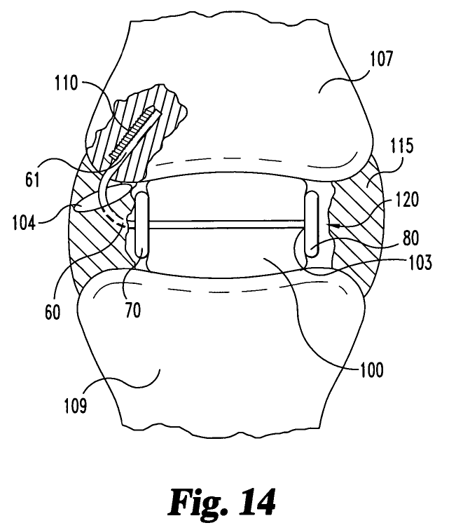

Referring now to FIGS. 12-14 in another form of the invention, a spinal implant system 120 is shown which includes spinal implant 100 and spinal implant anchoring device 50. Anchoring rod 60 extends through aperture, or defect, 104 of annulus fibrosis 115. Furthermore, first end 61 of anchoring rod 60 of the anchoring device is secured to upper vertebra 107, but may be secured to lower vertebra 109, or both upper and lower vertebrae, with an interference screw 110 as more fully described below and as shown in FIG. 14. As seen in FIG. 14, one end of the anchoring rod is wedged between the screw and the bone. Furthermore, first end 61 of anchoring device 50 may be secured to both vertebra 107 and 109 for added stability if first end 61 is appropriately configured as discussed above.

The interference screws described herein can be non-resorbable, resorbable and made form a wide variety of materials, including metals, ceramics, polymers and combinations thereof. Non-resorbable metallic materials include stainless steels, cobalt chrome alloys, titanium, titanium alloys, shape memory materials as described above, especially those exhibiting superelastic behavior and including metals, and alloys thereof. Resorbable materials include polylactide, polyglycolide, tyrosine-derived polycarbonate, polyanhydride, polyorthoester, polyphosphazene, bioactive glass, calcium phosphate, such as hydroxyapatite, and combinations thereof. The anchoring devices may also be anchored with other soft tissue anchors known in the art, including suture anchors commonly used in arthroscopy or sports medicine surgeries, for example. In the case of a soft tissue or suture anchor, the end of the elongated body of the anchoring device is attached to the end of the anchor, which is embedded and anchored in an adjacent vertebral body.

A wide variety of spinal implants for serving differing functions may be anchored with the anchoring devices described herein, including implants sized and configured for nucleus pulposus replacements, sized and configured for partial or full disc replacements or other disc reconstruction or augmentation purposes. Elastic, or otherwise resilient, implants are most preferred. For example, implants may be formed from hydrophilic materials, such as hydrogels, or may be formed from biocompatible elastomeric materials known in the art, including silicone, polyurethane, polyolefins such as polyisobutylene and polyisoprene, copolymers of silicone and polyurethane, neoprene, nitrile, vulcanized rubber and combinations thereof. In a preferred embodiment, the vulcanized rubber is produced by a vulcanization process utilizing a copolymer produced, for example, as in U.S. Pat. No. 5,245,098 to Summers et al., from 1-hexene and 5-methyl-1,4-hexadiene. Preferred hydrophilic materials are hydrogels. Suitable hydrogels include natural hydrogels, and those formed from polyvinyl alcohol, acrylamides such as polyacrylic acid and poly (acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrolidone, N-vinyl lactams, acrylamide, polyurethanes and polyacrylonitrile or may be formed from other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyetherurethane, polycarbonate-urethane and silicone polyether-urethane. Other suitable hydrophilic polymers include naturally-occurring materials such as glucomannan gel, hyaluronic acid, polysaccharides, such as cross-linked carboxyl-containing polysaccharides, and combinations thereof. The nature of the materials employed to form the elastic body should be selected so the formed implants have sufficient load bearing capacity. In preferred embodiments, a compressive strength of at least about 0.1 MPa is desired, although compressive strengths in the range of about 1 MPa to about 20 MPa are more preferred.

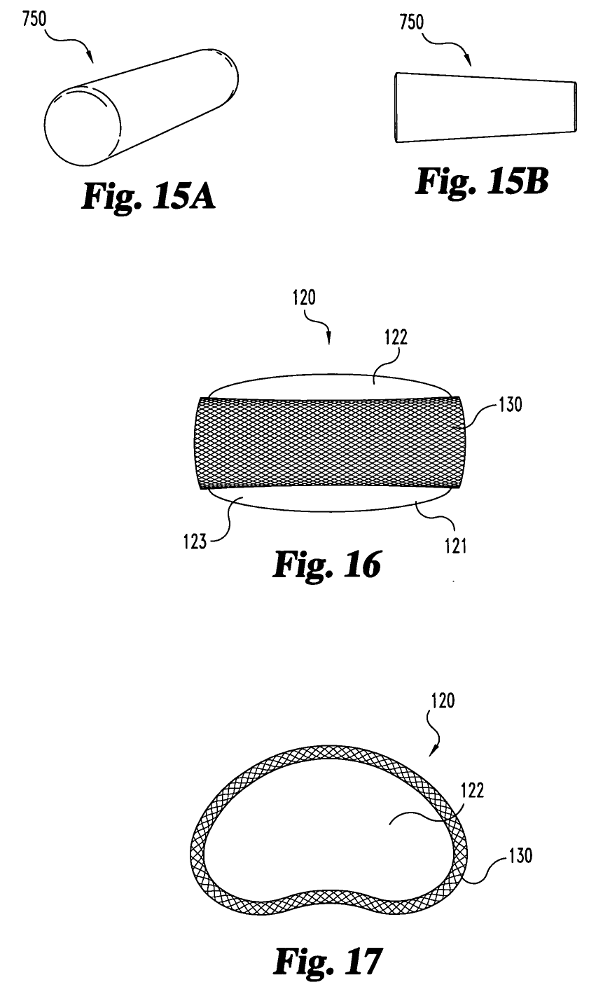

The implants can be shaped as desired. For example, the nucleus pulposus implants may take the form of a cylinder, a rectangle, or other polygonal shape or may be substantially oval. The implants may include elastic bodies 750 that are tapered, such as at one end, as seen in FIGS. 15A and 15B, in order to create or maintain lordosis. Furthermore, in certain forms of the invention, the implants generally conform to the shape of the nuclear disc space. Additionally, implants can be sized to fit within an intervertebral disc space, preferably surrounded by an annulus fibrosis, or at least partially surrounded by an annulus fibrosis. That is, the implants preferably are of a height and have a diameter that approximates the height and diameter of an intervertebral disc space. In certain forms of the invention, a spinal implant may be a nucleus pulposus implant and may thus be sized to fit within the natural intervertebral disc space. In other embodiments, the spinal implants may be disc replacements as described herein, and may be sized to fit within the intervertebral disc space that includes the space resulting when the inner annulus fibrosis layer, or a portion thereof, is removed. Such a spinal implant would therefore be sized to fit within the larger intervertebral disc space that includes the space resulting from removal of a portion of the annulus fibrosis, and would thus typically have a width or diameter that is substantially larger than the natural nucleus pulposus.

As mentioned above, the implant to be anchored preferably is reinforced for increased strength and to decrease lateral deformation of the implant. Accordingly, in yet another aspect of the invention, a reinforced spinal implant is provided. Referring now to FIGS. 16 and 17, implant 120 includes a load bearing elastic body 121 with an upper surface 122 and a lower surface 123. Implant 120 includes a preferably flexible, supporting member, such as peripheral supporting band 130 disposed circumferentially about body 121. Band 130 is similar to band 100 discussed above, with the exception that band 130 does not have openings therethrough on opposing sides of the band. As the implant, including the elastic body and supporting band, advantageously may replace all or a portion of the natural nucleus pulposus, while retaining the annulus fibrosis or a portion thereof, the implant may be sized to fit within the intervertebral disc space defined by the annulus fibrosis or a portion thereof.

As seen in FIG. 16, elastic body 121 includes upper and lower surfaces 122 and 123, respectively, portions of which are exposed to directly contact adjacent vertebral endplates. This exposure allows the lubricated upper and lower surfaces of elastic body 121 to articulate against the endplates to minimize abrasive wear of supporting band 130 and the endplates. Although the amount of the upper and lower surfaces of elastic body 121 that are exposed may vary, typically at least about 50%, preferably at least about 70%, more preferably at least about 80% and most preferably at least about 90% of the surfaces are exposed. In certain forms of the invention, the elastic body core may function as a nucleus pulposus, and thus functions as a load bearing component with stress transfer capabilities.

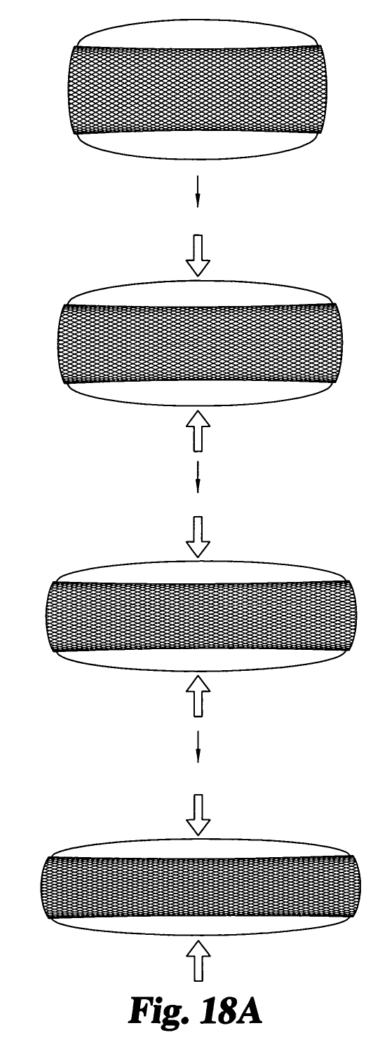

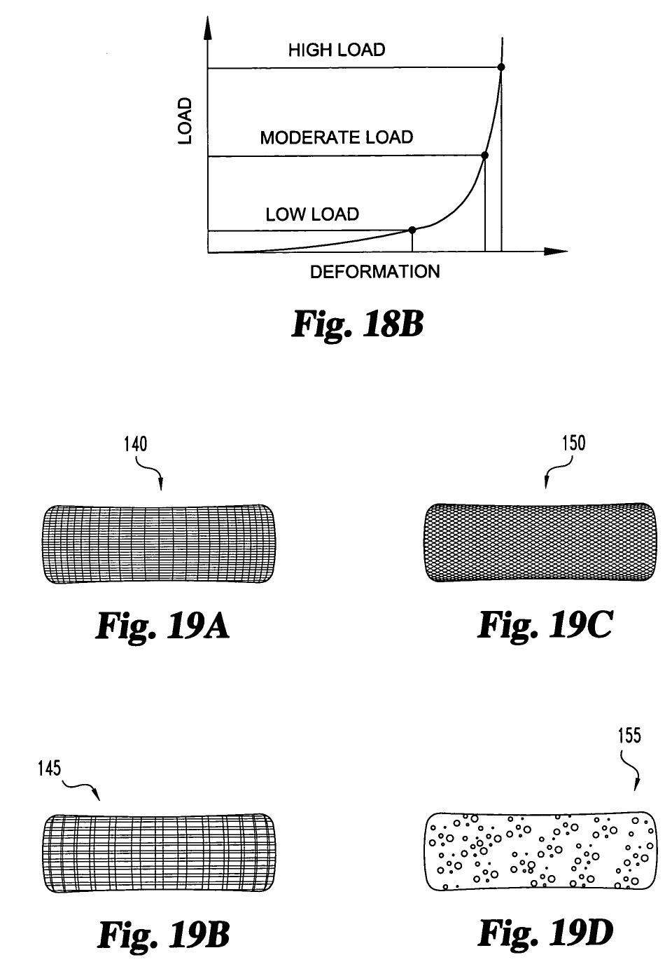

Peripheral supporting band 130 helps restrict excessive horizontal deformation of elastic body 121 upon loading conditions, as seen progressively in FIG. 18A, thereby helping to restore and maintain disc height. The hoop stress in the band increases exponentially after some small, initial deformation as seen in FIG. 18B. Band 130 preferably decreases lateral deformation, compared to deformation of an implant without the circumferential reinforcing band, as desired. Band 130 may, for example, decrease lateral deformation by at least about 20%, preferably at least about 40%, further preferably at least about 60%, more preferably at least about 80% and most preferably at least about 90%. An implant, such as one that includes an elastic body, having such a flexible supporting band, will be flexible and otherwise resilient to allow the natural movements of the disc and provides shock absorption capability at low to moderate applied stress, but will resist excessive deformation for disc height maintenance under high loading conditions. As described herein in the case of a lumbar disc, for example, low applied stress includes a force of about 100 Newtons to about 500 Newtons, moderate stress includes a force of about 500 Newtons to about 1000 Newtons, and high loading conditions, or high stress, includes a force of about above 1000 Newtons. Such a reinforced implant may be advantageously anchored with the anchoring devices described herein. Moreover, other outer covers, or jackets, as described in U.S. Pat. No. 5,674,295 may be utilized to reinforce implants to be anchored with the devices described herein. In preferred forms of the invention, the bands, jackets, or other outer covers or similar supporting members are flexible in that they may be folded or otherwise deformed, but are substantially inelastic so that the implant is more fully reinforced or otherwise supported.

Peripheral supporting band 130, as well as other outer covers, or jackets, may be made from a wide variety of biocompatible polymers, metallic materials, or combination of materials that form a strong but flexible support to prevent excessive lateral (horizontal) deformation of the core under increasing compressive loading. Suitable materials include non-woven, woven, braided, or fabric materials made from polymeric fibers including cellulose, polyethylene, polyester, polyvinyl alcohol, polyacrylonitrile, polyamide, polytetrafluoroethylene, polyparaphenylene terephthalamide, and combinations thereof. Other suitable materials include non-reinforced or fiber-reinforced elastomers such as silicone, polyolefins such as polyisobutylene and polyisoprene, polyurethane, copolymers of silicone and polyurethane, neoprene, nitrile, vulcanized rubber and combinations thereof. In a preferred form of the invention, a combination, or blend, of silicone and polyurethane is used. Furthermore, the vulcanized rubber is preferably produced as described above for the spinal implants. Supporting band 130 is advantageously made from materials described herein that allow it to be porous, which, in the case of an elastic body made from a hydrogel, or other hydrophilic material, allows fluid circulation through the elastic core body to enhance pumping actions of the intervertebral disc. Supporting members may further be formed from carbon fiber ceramic, ceramic fibers, metallic fibers, or other similar fibers described, for example, in U.S. Pat. No. 5,674,295, or from metallic materials that include shape memory materials as described above, especially those exhibiting superelastic behavior, titanium, titanium alloys, stainless steel, cobalt chrome alloys and combinations thereof. FIGS. 19A-19D show supporting bands of various patterns, including braided patterns (bands 140, 145 and 150) or porous patterns (band 155). It is realized that the braided materials may also be porous.

In addition to reinforcing the implants described herein with an outer cover, jacket or supporting band as described above, spinal implants 100, such as those formed from a hydrogel material, that are advantageously anchored with the anchoring devices described herein may be reinforced by forming the implant by molding hydrogels of different stiffness together and by annealing methods that include dipping the hydrogel in a hot oil bath, as described in U.S. Pat. No. 5,534,028. Other suitable reinforced spinal implants, such as nucleus pulposus implants, that may advantageously be used in the system of the present invention include those described in U.S. Pat. No. 5,336,551, as well as the novel implants described herein. As discussed above, the implant may be advantageously shaped to conform to the intervertebral disc space, or shaped as otherwise desired, as long as the implant has load bearing capability. Although the amount of load the implant is required to bear may vary depending on several factors, including the particular location in which the implant will be positioned, as well as the general health of the surrounding intervertebral discs, it is preferred that the implant be able to bear a load of at least about 20 Newtons for cervical discs, at least about 50 Newtons for thoracic discs and at least about 100 Newtons for lumbar discs.

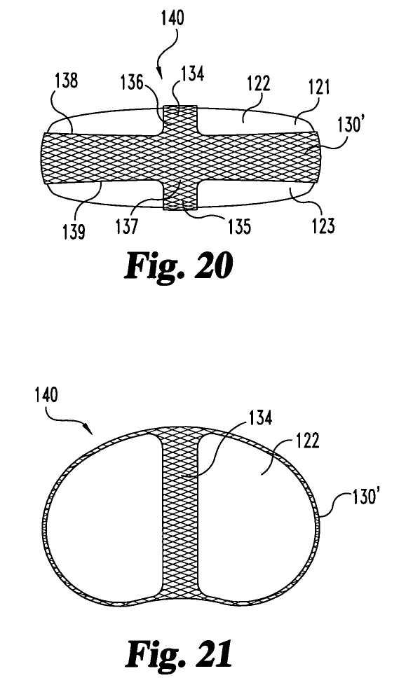

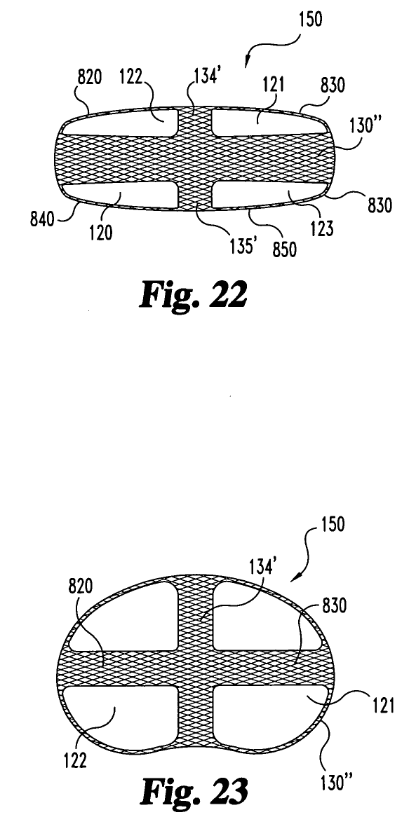

In yet other forms of the invention, an implant reinforced with a peripheral supporting band as described above is provided that is further reinforced with one or more straps. The straps may be advantageous in preventing the peripheral supporting band described herein from slipping, or otherwise sliding off the implant. Referring now to FIGS. 20 and 21, at least one strap 134 extends along upper surface 122 and at least one strap 135 extends along lower surface 123 of elastic body 121 of implant 140. Ends 136 of strap 134 and ends 137 of strap 135 are each preferably connected, or otherwise attached, to peripheral supporting band 130′. The point of attachment may be any location that will secure the strap, including at the upper margins 138 of the band, lower margins 139 of the band or any region between the upper and lower margins. Although two straps 134 and 135 are shown extending along upper surface 122 and lower surface 123, respectively, in FIGS. 20 and 21, one continuous strap may be utilized that extends completely around the implant, or the strap utilized may be in multiple pieces, as long as the combination of straps are sufficient to prevent excessive slipping and or sliding of the supporting band. Furthermore, more than one strap may extend along upper surface 122 and more than one strap may extend along lower surface 123. For example, as seen in FIGS. 22 and 23, straps 820, 830, 840 and 850 of implant 150 are attached to strap 130″. Straps 820 and 830 are also attached to strap 134′ and straps 840 and 850 are also attached to strap 135′.

In one preferred embodiment the peripheral band and/or the supporting strap(s) may be positioned in recessed grooves or channels in the elastic body to keep the band and/or strap(s) properly positioned. Additionally or alternatively, the surface of the elastic body may be concave to facilitate proper positioning of the supporting band(s) and/or strap(s).

When multiple bands and/or straps are used, the bands and/or straps may be positioned so that they are parallel to each other, or they may be positioned so that they intersect, as in an “x.” If the bands and/or straps intersect, the intersection point may be fixed or secured with stitches, adhesive, or another securing means, to keep the bands and/or straps properly positioned.

As mentioned above, the spinal implant with the flexible peripheral supporting band may be anchored utilizing the anchoring devices described in applicant's copending U.S. patent application Ser. No. 10/842,103. In other forms of the invention, implants as described herein may be anchored with an outer, preferably resorbable, shell as described in U.S. patent application Ser. No. 09/650,525 to Trieu, filed Aug. 30, 2000. In further forms of the invention, the implant may further include various outer surface features that may further restrain movement of the implant in the intervertebral disc space, with or without the outer shell. Such surface features are also more fully described in U.S. patent application Ser. No. 09/650,525 to Trieu, filed Aug. 30, 2000.

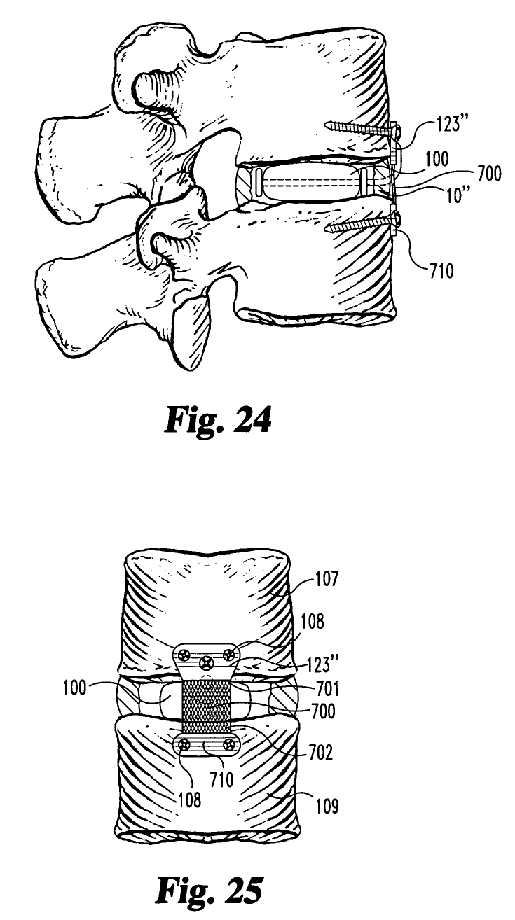

In yet other forms of the invention, a tension band 700 may be secured to the anchoring device and to an adjacent vertebra to, for example, provide further stabilization of the device, especially wherein the annulus and/or the ligament surrounding the annulus at the defect site are compromised. Referring now to FIGS. 24 and 25, one end 701 of band 700 may be attached to an anchoring device, such as anchoring device 10″ (similar to anchoring device 10 except that bracket 123″ is utilized), at, for example, bracket 123″, and the other end 702 may be secured to a plate 710, such as a metal plate, that is secured to the adjacent vertebra utilizing screws 108 as described herein. Band 700 may be attached to the anchoring device in a variety of ways, including crimping, tying, mechanical locking or may be secured with the same screws used to secure the anchoring device to the vertebral bodies. If two anchoring devices are utilized as described below, or if a single anchoring device is used that is secured to both adjacent vertebrae, one end 701 of tension band 700 may be attached to one of the brackets, or other areas, of the first anchoring device and the other end 702 of band 700 may be attached to the other bracket, or other area, of the second anchoring device. The tension band is preferably flexible to allow some degree of motion, but is substantially inelastic to prevent excessive extension.

The tension band may be formed from a wide variety of natural or synthetic tissue biocompatible materials. Natural materials include autograft, allograft and xenograft tissues. Synthetic materials include metallic materials and polymers. The metallic materials can be formed from shape memory alloy, including shape memory materials made from, for example, the nickel-titanium alloy known as Nitinol as described above. The shape memory materials may exhibit shape memory as described above, but preferably exhibit superelastic behavior. Other metallic materials include titanium alloy, titanium, stainless steel, and cobalt chrome alloy. Suitable polymeric materials include, for example, polyethylene, polyester, polyvinyl alcohol, polyacrylonitrile, polyamide, polytetrafluoroethylene, poly-paraphenylene, terephthalamide and combinations thereof. The materials used to form the tension band can be in a variety of forms, including the form of a fiber, woven, or non-woven fabric, braided, bulk solid and combinations thereof. The tension band may further be treated, such as by coating and/or impregnating, with bioactive materials that may enhance tissue ingrowth and/or attachment, including hydroxyapatite, bioglass, and growth factors. Suitable growth factors include transforming growth factors, insulin-like growth factors, platelet-derived growth factors, fibroblast growth factors, bone morphogenetic proteins as further described herein and combinations thereof.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

All references cited herein are indicative of the level of skill in the art and are hereby incorporated by reference in their entirety.

What is claimed is:

1. A spinal implant, comprising:
(a) an elastic body sized for introduction into an intervertebral disc space that is defined at least partially by an annulus fibrosis, said body having an upper surface and a lower surface for contacting adjacent vertebral endplates; and
(b) a flexible peripheral supporting band disposed circumferentially about said elastic body for reducing deformation of said body, at least a portion of said upper and lower surface free of said supporting band, said implant sized to fit within the intervertebral disc space defined by an annulus fibrosis.

2. The implant of claim 1, wherein said elastic body is comprised of a biocompatible polymeric material.

3. The implant of claim 1, wherein said material is comprised of a hydrogel.

4. The implant of claim 1, wherein said elastic body is comprised of an elastomer.

5. The implant of claim 4, wherein said elastomer is selected from silicone, polyurethane, copolymers of silicone and polyurethane, polyolefins, vulcanized rubber and combinations thereof.

6. The implant of claim 1, wherein said elastic body is comprised of a hydrogel/elastomer composite.

7. The implant of claim 1, wherein said band is made of a solid material.

8. The implant of claim 1, wherein said band is made of a porous material.

9. The implant of claim 1, wherein said band is made of a made of a woven material.

10. The implant of claim 1, wherein said band is made of a braided material.

11. The implant of claim 1, wherein said band is comprised of a biocompatible material selected from the group consisting of silicone, polyurethane, copolymers of silicone and polyurethane, polyolefins, vulcanized rubber, a shape memory material, stainless steel, titanium, titanium alloy, cobalt chrome alloy and combinations thereof.

12. The implant of claim 11, wherein said shape memory material is a shape memory alloy that exhibits superelastic behavior.

13. The implant of claim 1, wherein said peripheral supporting band is comprised of a fabric.

14. The implant of claim 13, wherein said fabric is selected from polyethylene, polyester, polyvinyl alcohol, polyacrylonitrile, polyamide, polytetrafluoroethylene, poly-paraphenylene terephthalamide, cellulose and combinations thereof.

15. The implant of claim 1, wherein at least about 50% of each of said upper and lower surface is free of said peripheral supporting band.

16. The implant of claim 1 wherein said flexible peripheral supporting band is disposed in a groove circumferentially about said elastic body.

17. The implant of claim 1, wherein said peripheral supporting band is elastic.

18. The implant of claim 1, wherein said implant includes at least one strap extending along said upper surface and at least one strap extending across said bottom surface.

19. The implant of claim 18, wherein at least one of said at least one straps is attached to said supporting band.

20. The implant of claim 18 wherein at least one of said at least one straps is positioned in a groove on the surface of said elastic body.

21. The implant of claim 18 wherein said at least one straps comprises at least two straps.

22. The implant of claim 21, wherein said at least two straps are positioned generally parallel to each other.

23. The implant of claim 21, wherein said at least two straps are positioned generally perpendicular to each other.
